(19) United States
(12) Patent Application Publication
(10) Pub. No.: US 2017/0368367 A1
(43) Pub. Date: Dec. 28, 2017

(54) LIGHT-EMITTING APPARATUS, LIGHT EMISSION METHOD, AND LIGHT-EMITTING SYSTEM

(71) Applicant: Panasonic Intellectual Property Management Co., Ltd., Osaka (JP)

(72) Inventors: Ayumu SATO, Niigata (JP); Hironori TAKESHITA, Osaka (JP)

(21) Appl. No.: 15/629,184

(22) Filed: Jun. 21, 2017

(30) Foreign Application Priority Data
Jun. 24, 2016 (JP) 2016-125736

(51) Int. Cl. A61N 5/06; H05B 37/02

(52) U.S. Cl. CPC A61N 5/062; H05B 37/0245; A61N 2005/0648; A61N 2005/0651

(57) ABSTRACT

A light emission apparatus emits light that activates a human body. The light emission apparatus includes a light-emitting module that emits light from a light emission region. The light emission region includes a first light emission region that emits light having a first luminance and a second light emission region that emits light having a second luminance greater than the first luminance. The second light emission region is a different region than the first light emission region. The first light emission region is located in a central region of the light emission region. The second light emission region is peripheral to the first light emission region. The light having the first luminance emitted from the first light emission region has a predetermined wavelength that activates the human body.

### CROSS REFERENCE TO RELATED APPLICATION

This application claims the benefit of priority of Japanese Patent Application Number 2016-125736 filed on Jun. 24, 2016, the entire content of which is hereby incorporated by reference.

### BACKGROUND

### 1. Technical Field

The present disclosure relates to a light-emitting apparatus that emits light for activating a human body, a light emission method of emitting light for light bathing using the light-emitting apparatus, and a light-emitting system including the light-emitting apparatus.

### 2. Description of the Related Art

Conventionally, light-emitting apparatuses including a light source (one example of the light-emitting module) are used in a variety of applications. For example, Japanese Unexamined Patent Application Publication No. H8-150210 discloses a high-illuminance light-therapy light-emitting apparatus (one example of the light-emitting apparatus) that reduces the luminance of the light emission surface (one example of the light emission region), which outputs light for therapeutic purposes (one example of the light for activating a human body), so as to output low-luminance, high-illuminance light (one example of the light).

However, even when exposed to light using an apparatus that simply reduces the luminance of the light emission region, the human body is not necessarily activated by the light. In order to activate the human body, the eyes of the user need to be exposed to the light, but if the luminance of the light is high, the pupils of the eyes contract thereby reducing the efficiency at which light enters the pupils.

In view of this, the present disclosure has an object to provide a light-emitting apparatus, light emission method, and light-emitting system capable of inhibiting a reduction in the efficiency at which light enters the pupils.

In order to achieve the above object, a light-emitting apparatus according to one aspect of the present disclosure is a light-emitting apparatus that emits light that activates a human body, and includes alight-emitting module that emits light from a light emission region. The light emission region includes: a first light emission region that emits light having a first luminance; and a second light emission region that emits light having a second luminance greater than the first luminance, the second light emission region being a different region than the first light emission region. The first light emission region is located in a central region of the light emission region, the second light emission region is peripheral to the first light emission region, and the light having the first luminance emitted from the first light emission region has a predetermined wavelength, that activates the human body.

Moreover, a light emission method according to one aspect of the present disclosure is a method of emitting light for light bathing using the light-emitting apparatus described above, and includes irradiating a human eye with light from the light emission region, the irradiating being performed by the light-emitting apparatus.

Moreover, a light-emitting system according to one aspect of the present disclosure includes: a terminal apparatus that transmits a signal; and a light-emitting apparatus that emits light that activates a human body. The light-emitting apparatus includes; a light-emitting module that emits light from a light emission region; a receiver that receives the signal from the terminal apparatus; and a controller that controls an illumination state of the light-emitting module in accordance with the signal received by the receiver. The light emission region includes: a first light emission region that emits light having a first luminance; and a second light emission region that emits light having a second luminance greater than the first luminance, the second light emission region being a different region than the first light emission region. The first light emission region is located in a central region of the light emission region, the second light emission region is peripheral to the first light emission region, and the light having the first luminance emitted from the first light emission region has a predetermined wavelength that activates the human body.

Accordingly, a reduction in the efficiency at which light enters the pupils can be inhibited.

### BRIEF DESCRIPTION OF DRAWINGS

The figures depict one or more implementations in accordance with the present teaching, by way of examples only, not by way of limitations. In the figures, like reference numerals refer to the same or similar elements.

FIG. 1 is a perspective view of the light emission apparatus according to Embodiment 1;

FIG. 2 is a cross sectional view of the light emission apparatus according to Embodiment 1, taken at II-II in FIG. 1;

FIG. 3 is a block diagram of the light emission apparatus according to Embodiment 1;

FIG. 4 is a front view of the light emission apparatus according to Embodiment 1;

FIG. 5 illustrates a spectrum of white light sources in the light emission apparatus according to Embodiment 1;

FIG. 6 illustrates a spectrum of white and blue light sources in the light emission apparatus according to Embodiment 1;

FIG. 7 is a front view of the light emission apparatus according to Variation 1 of Embodiment 1;

FIG. 8 is a front view of the light emission apparatus according to Variation 2 of Embodiment 1;

FIG. 9 is a perspective view of the light-emitting system according to Embodiment 2; and

FIG. 10 is a block diagram of the light emission apparatus in the light-emitting system according to Embodiment 2.

### DETAILED DESCRIPTION OF THE EMBODIMENTS

### (Underlying Knowledge Forming Basis of the Present Disclosure)

It is generally known that the human body activates when light of a certain wavelength enters the eyes. Light that activates the human body includes blue light and white light. In regard to blue light, exposure to light including blue light for a predetermined amount of time or longer is known to be effective in improving biological rhythm. In particular, exposure to blue light from noon to 3 p.m. is known to be effective in promoting the production of hormones such as serotonin. Moreover, in regard to white light, exposure to light including white light for a predetermined amount of time or longer is known to be effective in activating brain waves. Accordingly, it is desirable that the human body be activated by exposing the eyes to both blue and white light at the same time. Here, biological rhythm refers to a rhythm with a roughly 24-hour cycle in which a person naturally becomes sleepy at a certain time and naturally wakes up after sleeping for a certain amount of time as a physiological phenomenon.

Exposure to light each day increases the amount of serotonin secreted which promotes the production of melatonin, which affects the biological rhythm (biological clock). Since the production of melatonin is inhibited at night, deep sleep is possible.

For these reasons, effectively exposing human eyes to light is desirable. However, when the eyes are exposed to high-luminance light, a physiological phenomenon occurs whereby the pupils contract and the person naturally squints. When the pupils contract, the amount of light that enters the eyes decreases.

Regarding the sensitivity of the human eye, the human eye mainly perceives light whose wavelength is between from 380 nm to 780 nm and is most sensitive to light whose wavelength is 555 nm. The sensitivity decreases as the wavelength departs from this peak wavelength of 555 nm. Taking into account these characteristics (the spectral sensitivity) of the human eye, in order to increase the amount of blue light that enters the eyes while at the same time preventing a glaringly bright perception of the light, it is conceivable that reducing the luminance in wavelengths around 555 nm and increasing the luminance in wavelengths from 430 nm to 495 nm, i.e., blue light, would be effective.

Moreover, the sensitivity that has the greatest effect on human hormones (also referred to as biological effect level or biological stimulation level) peaks between from 480 nm to 490 nm. Accordingly, it is desirable that light entering the human eye in wavelengths from 480 nm to 490 nm be high in intensity.

In view of this, when the luminance of the light emission region is kept low like with the conventional light-emitting apparatus, the pupils tend not to contract, but merely keeping the luminance of the light emission region low does not necessarily activate the human body. It is desirable to expose the eyes to (introduce into the eyes) light that effectively activates the human body while inhibiting a reduction in the efficiency at which light enters the pupils.

The present disclosure provides a light-emitting apparatus, light emission method, and light-emitting system capable of inhibiting a reduction in the efficiency at which light enters the pupils.

The following describes embodiments with reference to the drawings. Note that the embodiments described below each show a specific example of the present disclosure. The numerical values, shapes, materials, elements, the arrangement and connection of the elements, etc., indicated in the following embodiments are mere examples, and therefore do not intend to limit the inventive concept. Therefore, among elements in the following embodiments, those not recited in any of the independent claims defining the most generic part of the inventive concept are described as optional elements.

Note that the figures are schematic diagrams and are not necessarily precise illustrations. Additionally, like reference signs indicate like elements in the figures. As such, overlapping explanations of like elements are omitted or simplified.

Hereinafter, a light emission apparatus according to Embodiment 1 of the present disclosure will be described.

### Embodiment 1

### (Configuration)

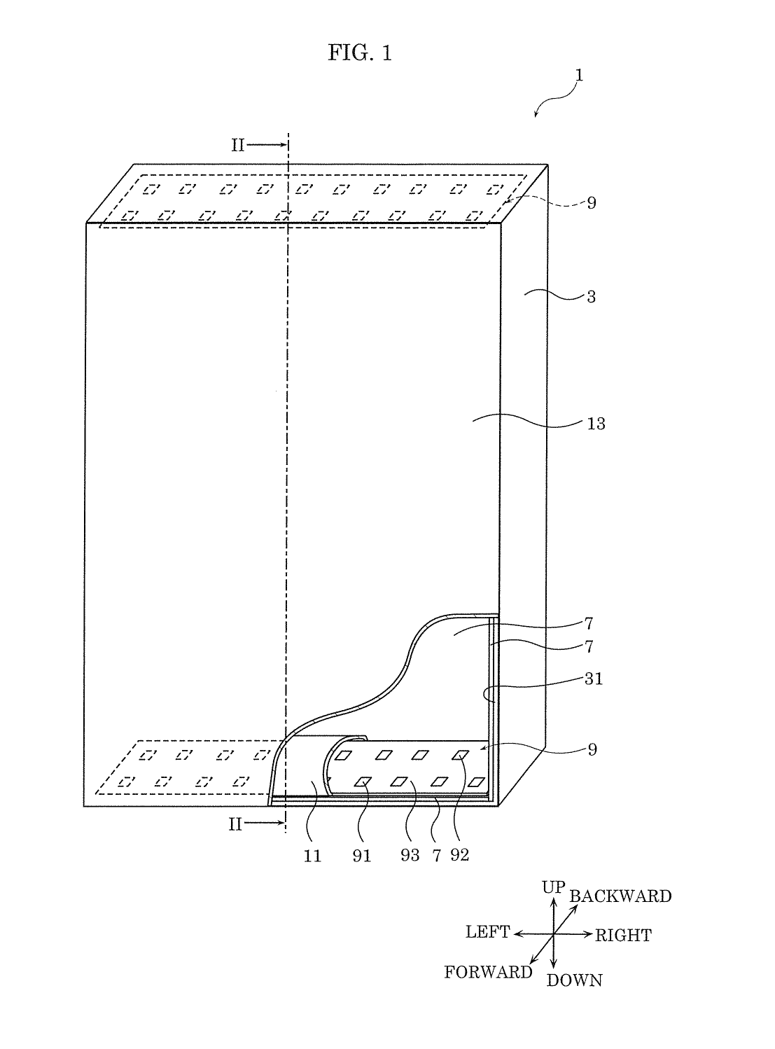

First, light emission apparatus 1 according to this embodiment will be described with reference to FIG. 1 through FIG. 4.

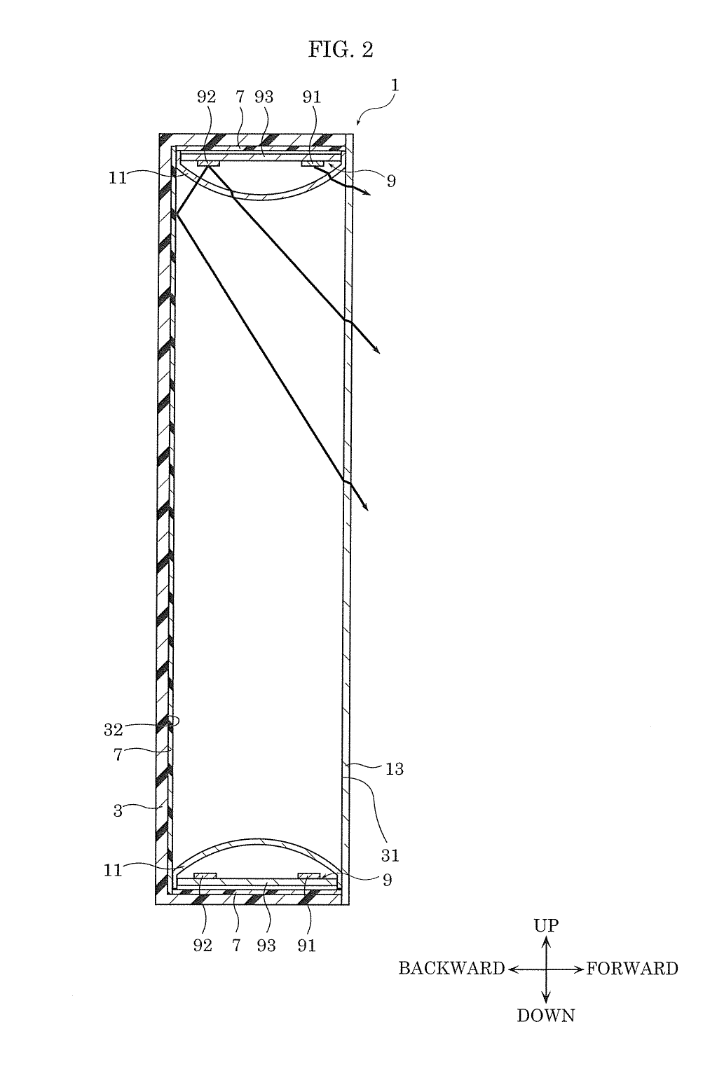

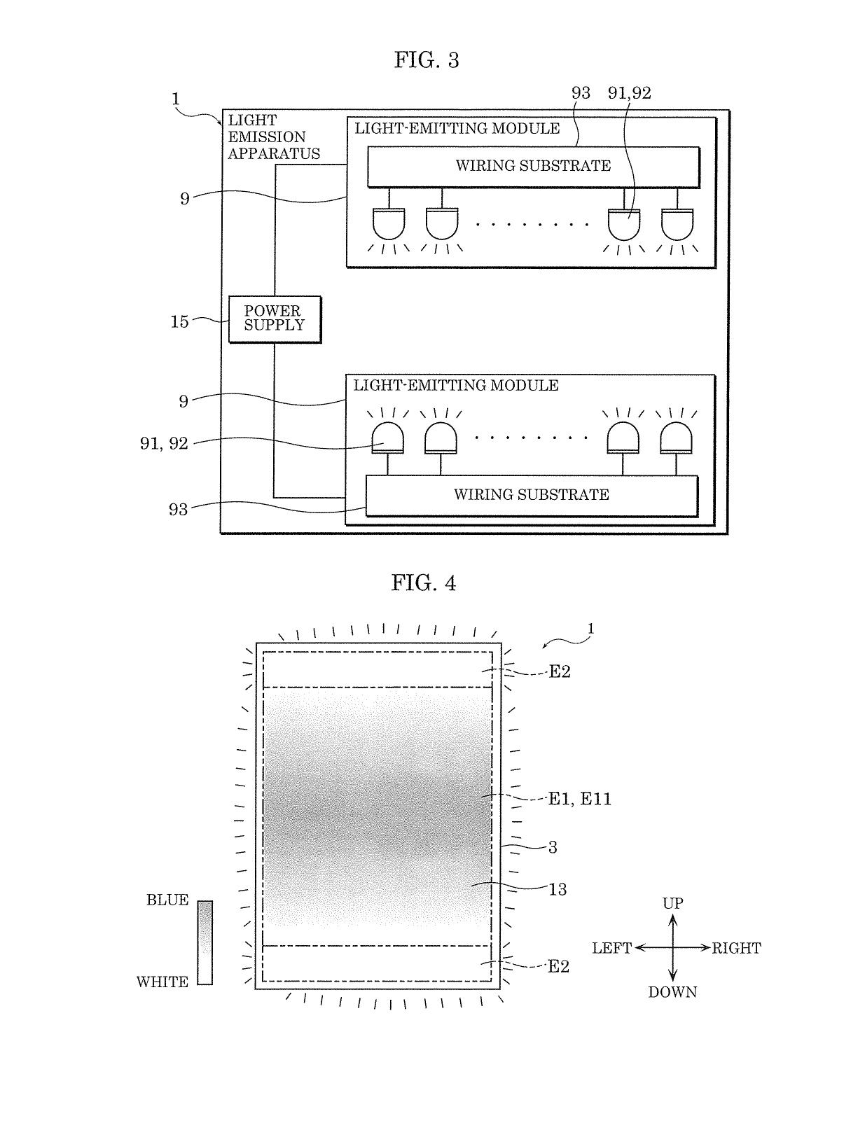

FIG. 1 is a perspective view of light emission apparatus 1 according to Embodiment 1. FIG. 2 is a cross sectional view of light emission apparatus 1 according to Embodiment 1, taken at line II-II in FIG. 1. FIG. 3 is a block diagram of light emission apparatus 1 according to Embodiment 1. FIG. 4 is a front view of light emission apparatus 1 according to Embodiment 1. In FIG. 2, the bold, solid line arrows indicate rays of blue and white light.

FIG. 1 illustrates that in light emission apparatus 1, the side of light emission apparatus 1 on which light-transmissive plate 13 is disposed is the front side of light emission apparatus 1. FIG. 1 illustrates the relative front, back, left, right, up, and down directions. The directionality of FIG. 2 and all subsequent figures corresponds to the directionality illustrated in FIG. 1. Note that in FIG. 1, the up and down directions, the left and right directions, and the forward and backward directions vary depending on usage, and are therefore not limited to this example. The same applies to the subsequent figures as well.

As illustrated in FIG. 1, light emission apparatus 1 (one example of the light-emitting apparatus) emits, from the front toward the user, light that activates the human body. Light that activates the human body is, in this embodiment, light including a large amount of blue light and light consisting of blue light added with white light. In this embodiment, blue and white light can be emitted at the same time. For example, light emission apparatus 1 can be placed on a desk in, for example, an office, or on a vanity table, and light that activates the user's body can be emitted.

Note that here, “blue light” does not strictly refer to the color blue, but refers to light that is commonly perceived as being blue, and refers to, for example, light whose wavelength is between from 430 nm to 495 nm. Moreover, “white light” does not strictly refer to the color white, but refers to light that is commonly perceived as being white, and refers to light consisting of an even mixture of visible light rays of all colors (for example, blue light (430 nm to 495 nm), green light (495 nm to 570 nm), and red light (620 nm to 750 nm)).

As illustrated in FIG. 2 and FIG. 3, light emission apparatus 1 includes enclosure 3, five diffusion sheets 7, a pair of light-emitting modules 9, two light-transmissive covers 11, light-transmissive plate 13, and power supply 15.

Enclosure 3 is a box that has a bottom and has a low profile in the front-back direction. Enclosure 3 houses the five diffusion sheets 7, the pair of light-emitting modules 9, and power supply 15. In this embodiment, enclosure 3 has, in a front view, a rectangular shape that is elongated in the up-down direction. In this embodiment, enclosure 3 has a height of 210 mm in the up-down direction and a width of 150 mm in the left-right direction.

Moreover, enclosure 3 includes opening 31 that opens on the front (front surface side). Diffusion sheets 7, which are white in color and diffuse (reflect) light, are provided on all inner surfaces of enclosure 3. More specifically, diffusion sheets 7 are provided so as to cover the back, upper, lower, left, and right inner surfaces of enclosure 3. In this embodiment, enclosure 3 does not have a front surface, but when enclosure 3 does have a front surface, diffusion sheet 7 may be provided so as to cover the front inner surface.

Diffusion sheets 7 are made of, for example, a material having a light transmitting property, such as light transmissive resin—examples of which include acrylic or polycarbonate—or clear glass. Moreover, in this embodiment, diffusion sheets 7 have a function of diffusing light. For example, a milky-white light diffusion film is formed on each diffusion sheet 7 by adhering, to the inner or outer surface of each diffusion sheet 7, a resin containing a light diffusing material (particles)—such as silica or calcium carbonate—or white pigment. Moreover, each diffusion sheet 7 itself may be formed using, for example, a resin material dispersed with a light diffusing material.

Diffusion sheets 7 may be configured so as to have a light diffusing property by treating diffusion sheets 7 with a light diffusion treatment. For example, the surface of each diffusion sheet 7 may be texture treated by forming fine dimples in the surface, and, alternatively, diffusion sheets 7 may be configured so as to have a light diffusing property by printing a dot pattern on the front surface of a transparent cover.

Note that diffusion sheets 7 are not limited to a material having a light transmitting property (a transparent or semitransparent material). Diffusion sheets 7 may include a metal material such as aluminum, and may include a hard white resin material (opaque resin). In other words, it is acceptable so long as diffusion sheets 7 have a function of diffusing (reflecting) light.

Each light-emitting module 9 includes a plurality of light sources (white light sources 91 and blue light sources 92; to be described later) and wiring substrate 93 on which the plurality of light sources are disposed. Each light-emitting module 9 has a plate-like shape that is elongated in the left-right direction.

The plurality of light sources are mounted in one or more rows on wiring substrate 93. Each light source is a surface mount device (SMD) light-emitting diode (LED) element. An SMD LED element is, more specifically, a package LED element in which an LED chip (light-emitting element) is mounted in a resin-formed cavity sealed with a phosphor-containing resin. Each light source is turned on and off under control by a control circuit included in light emission apparatus 1. Moreover, the control circuit controls power supply 15 (adjusts the amount of power supplied) to control the dimming and color of each light source. In this embodiment, the dimming (brightness) and color (of emitted light) of light-emitting modules 9 may controllable. The control circuit is realized as, for example, a microcomputer, processor, or dedicated circuit that controls, for example, the value of current supplied to light-emitting modules 9 in accordance with an input signal.

Note that the light sources are not limited to the above example; a chip-on-board (COB) module in which LED chips are directly mounted to wiring substrate 93 may be used. Moreover, the light-emitting element included in each light source is not limited to an LED element; for example, the light-emitting element may be a semiconductor light-emitting element such as a semiconductor laser, or some other solid-state element such as an organic electroluminescent (EL) or inorganic EL element.

The light sources are aligned in two rows counting in the front-back direction. The light sources are disposed so as to be evenly spaced apart in the lengthwise (left-right) direction of wiring substrate 93. The light sources mounted in the front row extending in the left-right direction are white light sources 91 that emit white light. The light sources mounted in the back row extending in the left-right direction (behind white light sources 91) are blue light sources 92 that emit blue light. In this embodiment, white light sources 91 and blue light sources 92 are mounted on wiring substrate 93 such that their optical axes are parallel to one another.

A first (the upper one) of the pair of light-emitting modules 9 is disposed on the upper surface in enclosure 3. The second (the lower one) of the pair of light-emitting modules 9 is disposed on the lower surface in enclosure 3. The first light-emitting module 9 and the second light-emitting module 9 face each other. More specifically, in enclosure 3, white light sources 91 and blue light sources 92 on the upper light-emitting module 9 and white light sources 91 and blue light sources 92 on the lower light-emitting module 9 face each other.

Note that in this embodiment, the pair of light-emitting modules 9 are disposed so as to be parallel to each other, but the pair of light-emitting modules 9 need not be parallel to each other. For example, the upper light-emitting module 9 may slope downward from the front to the back (such that blue light sources 92 are disposed lower than white light sources 91), and the lower light-emitting module 9 may slope upward from the front to the back (such that white light sources 91 are disposed lower than blue light sources 92).

The two light-transmissive covers 11 are fixed to enclosure 3 so as to cover the pair of light-emitting modules 9 in one-to-one correspondence. Light-transmissive covers 11 are elongated in the left-right direction and have a light transmitting property, and in a cross section in a plane extending in the front-back and up-down directions, have a curve forming an arc.

The two light-transmissive covers 11 may be made from the same material as diffusion sheets 7, may have the same light diffusing function has diffusion sheets 7, and may be treated with the same light diffusion treatment as diffusion sheets 7 to impart the light diffusing property.

Light-transmissive plate 13 is a flat plate having a light transmitting property. Light-transmissive plate 13 is rectangular in shape and is equal in size to the front side of enclosure 3. Light-transmissive plate 13 is fixed to the front edges of enclosure 3 so as to cover opening 31 of enclosure 3. Light-transmissive plate 13 has a function of diffusing light emitted by white light sources 91 and blue light sources 92. Light-transmissive plate 13 may also be made from the same material as diffusion sheets 7, may have the same light diffusing function has diffusion sheets 7, and may be treated with the same light diffusion treatment as diffusion sheets 7 to impart the light diffusing property.

Power supply 15 is configured of a power circuit that generates power for causing the pair of light-emitting modules 9 to emit light. For example, power supply 15 converts power supplied from a power system to DC power of a predetermined level by, for example, rectifying, smoothing, and stepping down the power, and supplies the DC power to the pair of light-emitting modules 9. Power supply 15 is electrically connected to the power system via a power line such as a control line.

Power supply 15 turns ON and OFF the supply of power to the pair of light-emitting modules 9 under control by the control circuit. For example, when an ON operation is received via a control such as a remote control, the control circuit supplies power from power supply 15 to the pair of light-emitting modules 9 to turn on the pair of light-emitting modules 9. Moreover, when an OFF operation is received by the control, the control circuit interrupts the supply of power from power supply 15 to the pair of light-emitting modules 9 to turn off the pair of light-emitting modules 9.

Note that in light emission apparatus 1, one power supply 15 may be used to turn on white light sources 91 and blue light sources 92, and, alternatively, two power supplies 15 may be used, one to turn on white light sources 91 and one to turn on blue light sources 92. When two power supplies 15 are be used, one to turn on white light sources 91 and one to turn on blue light sources 92, dimming and color control may be performed by operation of the control connected to power supplies 15.

As illustrated in FIG. 4, with light emission apparatus 1 configured as described above, when the white light sources 91 and the blue light sources 92 of the upper and lower light-emitting modules 9 are turned on, the upper and lower end regions of light-transmissive plate 13 appear white in color and the central region (the region excluding the upper and lower end regions) of light-transmissive plate 13 appears blue in color. More specifically, light-transmissive plate 13 has a gradation such that the white color at the upper and lower end regions of light-transmissive plate 13 gradually changes to blue with increasing proximity to the central region. The region of light-transmissive plate 13 from which light is emitted is referred to as the light emission region. In this embodiment, the front surface of light-transmissive plate 13 (the surface of light-transmissive plate 13 from which light is emitted) is the light emission region.

The light emission region includes first light emission region E1 and second light emission regions E2.

First light emission region E1 is the central region of light-transmissive plate 13 from which blue light is emitted. Light having a first luminance of less than 23000 cd/m² is emitted from first light emission region E1. The first luminance of first light emission region E1 is at least 1000 cd/m² and less than 23000 cd/m² in order to inhibit the user from perceiving first light emission region E1 as being glaringly bright (inhibit the pupils from contracting) when the user looks directly at the light emitting from first light emission region E1. In particular, the first luminance in the central region of first light emission region E1 between the upper and lower second light emission regions E2 is desirably approximately 5000 cd/m². Moreover, the second luminance of second light emission regions E2 is at least 23000 cd/m² and at most 74000 cd/m².

First light emission region E1 includes gradation region E11. In gradation region E11, the first luminance of first light emission region E1 gradually decreases with increasing distance from second light emission regions E2. In other words, gradation region E11 is located between second light emission regions E2, which are regions other than first light emission region E1 and gradation region E11.

Note that gradation region E11 may be omitted and the luminance at the boundaries between first light emission region E1 and second light emission regions E2 may change abruptly. In other words, in this embodiment, gradation region E11 is not necessarily required. Note that gradation region E11 may be the entire first light emission region E1, and, alternatively, may be a portion of first light emission region E1.

Moreover, although first light emission region E1 includes gradation region E11, second light emission regions E2 may include gradation regions.

Second light emission regions E2 are contiguous with a portion of an outer boundary of the light emission region. More specifically, second light emission regions E2 sandwich first light emission region E1. In this embodiment, there are two second light emission regions E2, an upper second light emission region E2 and a lower second light emission region E2, which emit white light. Second light emission regions E2 are different regions from first light emission region E1, and are peripheral to first light emission region E1. Light having a second luminance greater than the first luminance is emitted from second light emission regions E2. In this embodiment, the second luminance is at least 23000 cd/m² and at most 50000 cd/m². The luminance at the borders between first light emission region E1 and second light emission regions E2 is 23000 cd/m², which is bearable even if the user looks directly at the emitted light.

Moreover, the pair of second light emission regions E2 appear white in color, and this is due to white light sources 91 being located closer to light-transmissive plate 13 than blue light sources 92. Accordingly, as illustrated by the bold, solid lines in FIG. 2, mainly white light is emitted from the upper and lower second light emission regions E2 of light-transmissive plate 13. On the other hand, first light emission region E1 (the central region) appears blue in color conceivably due to the percentage of the blue light being greater than the white light when the blue and white light mix together. In other words, the light emission region has a gradation such that the color becomes whiter from first light emission region E1 toward second light emission regions E2. Stated differently, the light emission region has a gradation such that the color becomes bluer with increasing distance from second light emission regions E2. As illustrated in FIG. 4, blue light is mainly emitted from first light emission region E1 in light-transmissive plate 13, whereby first light emission region E1 appears visually similar to a blue sky.

Viewing angle has been taken into consideration with light emission apparatus 1. More specifically, it is known that human vision includes a distinguishing field of view, an effective field of view, and a steady gaze field of view. The distinguishing field of view is a range in which visual performance, such as eyesight, is exceptional, and is a range within approximately ±2° along the horizontal axis from the center of the human eye (for example, the pupil) as a reference point. The effective field of view is a range in which information can be received instantaneously via eye movement, and is a range within approximately ±15° along the horizontal axis, within approximately 8° upward, and within approximately 12° downward from the center of the human eye (for example, the pupil) as a reference point. The steady gaze field of view is a range in which information can be received reasonably via eye and head movement, and is a range within approximately ±30° to ±45° along the horizontal axis, within approximately 20° to 30° upward, and within approximately 25° to 40° downward from the center of the human eye (for example, the pupil) as a reference point

Therefore, in light emission apparatus 1, the region from which blue light is emitted (first light emission region E1) is, for example, of a size that covers at least the range of the distinguishing field of view of the user. In other words, first light emission region E1 is, for example, of a size that is greater than the range of the distinguishing field of view. In view of the above, when the range of the distinguishing field of view is expressed as S, the distance between first light emission region E1 of light emission apparatus 1 and an eye of the user is expressed as D, and the angle of the distinguishing field of view is expressed as θ, the relationship between S, D, and θ can be expressed by the following formula.

S=D tan θ

For example, when the distance D between first light emission region E1 of light emission apparatus 1 and an eye of the user is 50 cm, and the angle θ of the distinguishing field of view is 2°, the range S of the distinguishing field of view is approximately 1.75 cm. In other words, first light emission region E1 (the central region of light-transmissive plate 13) needs to be larger than a circle 1.75 cm in diameter. Therefore, the region from which light having a luminance of 5000 cd/m² is emitted is desirably larger than a circle 1.75 cm in diameter. Note that in this embodiment, the region from which light having a luminance of 5000 cd/m² is emitted has height of approximately 2 cm in the up-down direction and a width of 150 mm in the left-right direction.

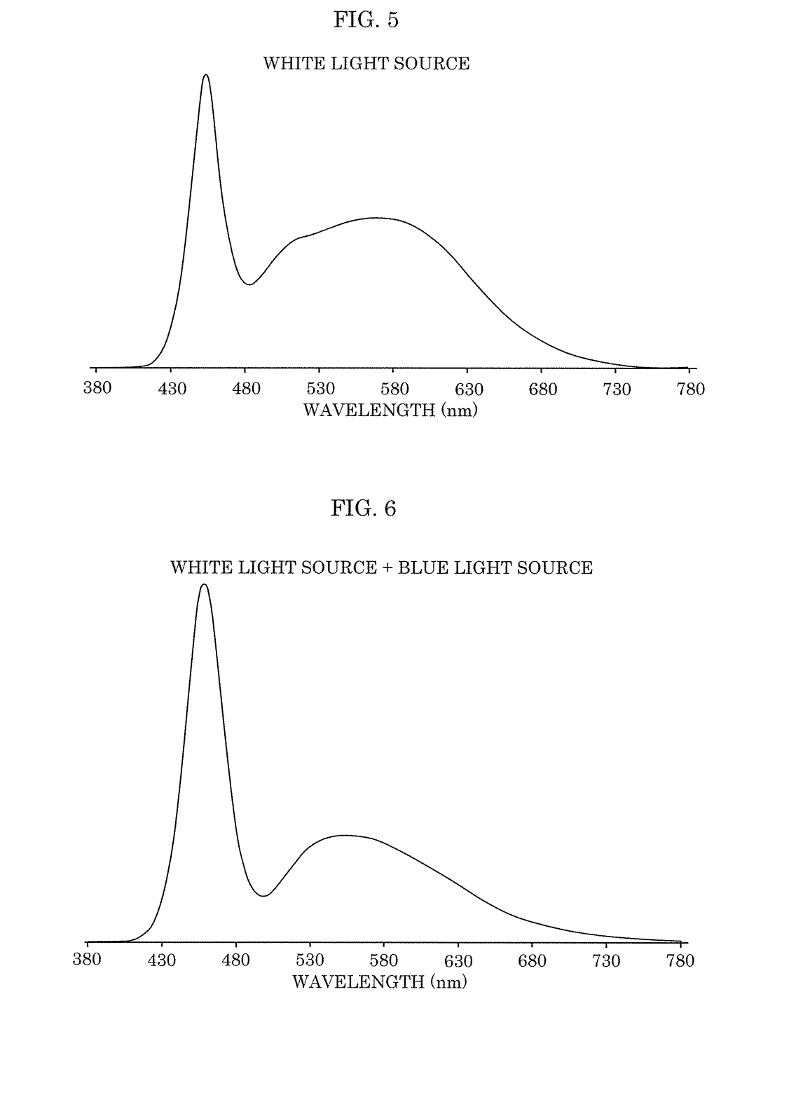

Next, the relationship between activation of a human body and light wavelength will be described. FIG. 5 illustrates a spectrum of white light sources 91 in light emission. apparatus 1 according to Embodiment 1. FIG. 6 illustrates a spectrum of white light sources 91 and blue light sources 92 in light emission apparatus 1 according to Embodiment 1.

As illustrated in FIG. 5, in the spectrum for white light sources 91, the peak wavelength of the blue light is 460 nm. The amount of blue light that effects human hormones may be proactively increased and light of wavelengths longer than approximately 495 nm (for example, yellow and red light) may be decreased. In other words, with light emission apparatus 1, the glaring brightness of the white light may be inhibited and the amount of blue light may be proactively increased. Accordingly, compared to when only white light is emitted from light emission region (FIG. 5), the amount of blue light can be increased, as illustrated in FIG. 6, by emitting blue light from first light emission region E1 and emitting white light from second light emission regions E2. Even when the peak wavelength of blue light is 460 nm, as is the case in this embodiment, an advantageous effect of activation of the human body can be expected by increasing the amount of blue light. Note that the spectrum illustrated in FIG. 6 is a non-limiting example. For example, the peak wavelength of the blue light illustrated in FIG. 6 may fall between 480 nm and 490 nm by using a light source that mainly emits light of wavelengths between 480 nm and 490 nm.

Next, a light emission method which uses light emission apparatus 1 will be described.

The user places light emission apparatus 1 a predetermined distance away from his or her eyes. The user supplies light emission apparatus 1 with power from a power system to operate light emission apparatus 1. Blue and white light is emitted from light emission apparatus 1. In this way, the user exposes him or herself to the blue light emitted from first light emission region E1 and the white light emitted from second light emission regions E2 at the same time.

Regarding the relationship between activation of a human body and light intensity, exposure to light having a predetermined illuminance for a predetermined amount of time is known to activate the secretion of the sleep hormone melatonin in the human body. Examples of known desirable exposure times and illuminance values for users of light emission apparatus 1 include: exposure of light with an illuminance of 5000 1× for approximately 6 hours (total of 30000 1×/h) in the case of a healthy adult male; exposure of light with an illuminance of 2500 1× for approximately 4 hours (total of 10000 1×/h) in the case of an elderly person with insomnia; and exposure of light with an illuminance of 5555 1× for approximately 6 hours (total of 33330 1×/h) in the case of a healthy adult female. In this way, the user can anticipate that his or her body will be activated if the user exposes him or herself to light every day using light emission apparatus 1.

### (Advantageous Effects)

Next, the advantageous effects of light emission apparatus 1 according to this embodiment will be described.

As described above, light emission apparatus 1 emits light that activates the human body. Light emission apparatus 1 includes light-emitting modules 9 that emit light from a light emission region. The light emission region includes first light emission region E1 that emits light having a first luminance and second light emission region E2 that emits light having a second luminance greater than the first luminance. Second light emission region E2 is a different region than first light emission region E1. First light emission region E1 is located in the central region of the light emission region. Second light emission region E2 is peripheral to first light emission region E1. The light having the first luminance emitted from first light emission region E1 has a predetermined wavelength that activates the human body.

With this configuration, since second light emission region E2 is peripheral to first light emission region E1, the user's gaze moves away from second light emission region E2 and toward the light whose luminance is lower than the second luminance. As such, the user's gaze moves toward first light emission region E1 from which light having the first luminance is emitted, whereby light of the first luminance more easily enters the eyes. In other words, the range of the distinguishing field of view fits within first light emission region E1. Moreover, with light emission apparatus 1, since the user's gaze shifts, light emitted from second light emission region E2 can be prevented from causing the pupils to contract and reduce the efficiency at which light enters the pupils.

Accordingly, the user exposes him or herself to light using light emission apparatus 1, a reduction in the efficiency at which light enters the pupils can be inhibited.

With this embodiment, since the user's gaze moves away from second light emission region E2 and toward first light emission region E1, blue and white light emitted from first light emission region E1 easily enters the eyes, whereby light having the first luminance and the second luminance and of a predetermined wavelength that activates the human body easily enters the eyes.

As described above, the light emission method according to this embodiment is a method of emitting light for light bathing using light emission apparatus 1. Light emission apparatus 1 irradiates the eyes with light from the light emission region.

With this method, if the user exposes him or herself to light using light emission apparatus 1, the same advantageous effects achieved with light emission apparatus 1 can be achieved.

Moreover, in light emission apparatus 1 according to this embodiment, second light emission regions E2 sandwich first light emission region E1.

With this configuration, even if the user's gaze moves away from first light emission region E1, the user's gaze can easily return to first light emission region E1, which is lower in luminance than second light emission regions E2. Accordingly, with light emission apparatus 1, a reduction in the efficiency at which light enters the pupils can be inhibited.

Moreover, light emission apparatus 1 according to this embodiment further includes enclosure 3 that houses the pair of light-emitting modules 9, and light-transmissive plate 13 that includes the light emission region and covers opening 31 in enclosure 3. Each light-emitting module in the pair of light-emitting modules 9 includes white light source 91 and blue light source 92. In enclosure 3, a first light-emitting module among the pair of light-emitting modules 9 and a second light-emitting module among the pair of light-emitting modules 9 face each other. Blue light sources 92 are disposed closer to bottom 32 of enclosure 3 than white light sources 91 are.

Compared to when white light sources 91 and blue light sources 92 are disposed on bottom 32 of enclosure 3 such that their optical axes all extend forward, with the configuration of light emission apparatus 1 according to this embodiment, the number of blue light sources 92 and white light sources 91 used can be reduced by disposing the pair of light-emitting modules 9 so as to face each other. Moreover, even if the pair of light-emitting modules 9 are disposed so as to face each other like they are in light emission apparatus 1 according to this embodiment, blue light can be emitted from first light emission region E1 and white light can be emitted from second light emission regions E2. Accordingly, with light emission apparatus 1, it is possible to prevent a steep rise in cost due to an increase in the number of components used.

Note that exposure to light including blue light for a predetermined amount of time or longer is generally known to be effective in improving biological rhythm. Moreover, exposure to light including white light for a predetermined amount of time or longer is generally known to be effective in activating brain waves. Accordingly, with light emission apparatus 1, it is possible to activate the human body by irradiating the eyes with light having a first luminance and a second luminance and having a predetermined wavelength.

Moreover, light emission apparatus 1 according to this embodiment further includes diffusion sheets 7 that are disposed on inner surfaces of enclosure 3 and diffuse light.

With this configuration, since blue light sources 92 are disposed closer to diffusion sheet 7 disposed on bottom 32 of enclosure 3 than white light sources 91 are, blue light is easily diffused. Accordingly, blue light is easily emitted from first light emission region E1.

Moreover, in light emission apparatus 1 according to this embodiment, the first luminance of first light emission region E1 is less than 23000 cd/m² and the second luminance of second light emission regions E2 is at least 23000 cd/m².

With this configuration, since the boundary between luminances in first light emission region E1 and second light emission regions E2 is 23000 cd/m², even if the user looks directly at the emitted light, the brightness is bearable (i.e., the user does not get an unpleasant feeling even if he or she looks directly at first light emission region E1). Accordingly, if the first luminance is less than 23000 cd/m², the pupils of the user tend not to contract. As a result, with light emission apparatus 1, a reduction in the efficiency at which light enters the pupils can be inhibited.

Moreover, in light emission apparatus 1 according to this embodiment, the predetermined wavelength includes a light emission peak of light-emitting modules 9 in a range from 430 nm to 495 nm. Moreover, the first luminance of first light emission region E1 is at least 1000 cd/m² and less than 23000 cd/m². The second luminance of second light emission regions E2 is at least 23000 cd/m² and at most 74000 cd/m².

With this configuration, even if the user looks directly at the light emitted from first light emission region E1, the user will not perceive the light as being glaringly bright. Accordingly, with light emission apparatus 1, it is possible to inhibit a reduction in the efficiency at which light enters the pupils, as well as to activate the human body (correct biological rhythm and activate brain waves) by irradiating the eyes with light having a first luminance and a second luminance and having a predetermined wavelength.

Moreover, in light emission apparatus 1 according to this embodiment, first light emission region E1 includes gradation region E11. In gradation region E11, the first luminance of first light emission region E1 gradually decreases with increasing distance from second light emission regions E2.

With this configuration, first light emission region E1 has a gradation such that the color becomes bluer with increasing distance from second light emission regions E2. Accordingly, first light emission region E1 appears visually similar to a blue sky, which comforts the user.

Moreover, in light emission apparatus 1 according to this embodiment, the color of the light from the first emission region is blue and the color of the light from the second emission region is white.

### Variation 1 of Embodiment 1

### (Configuration)

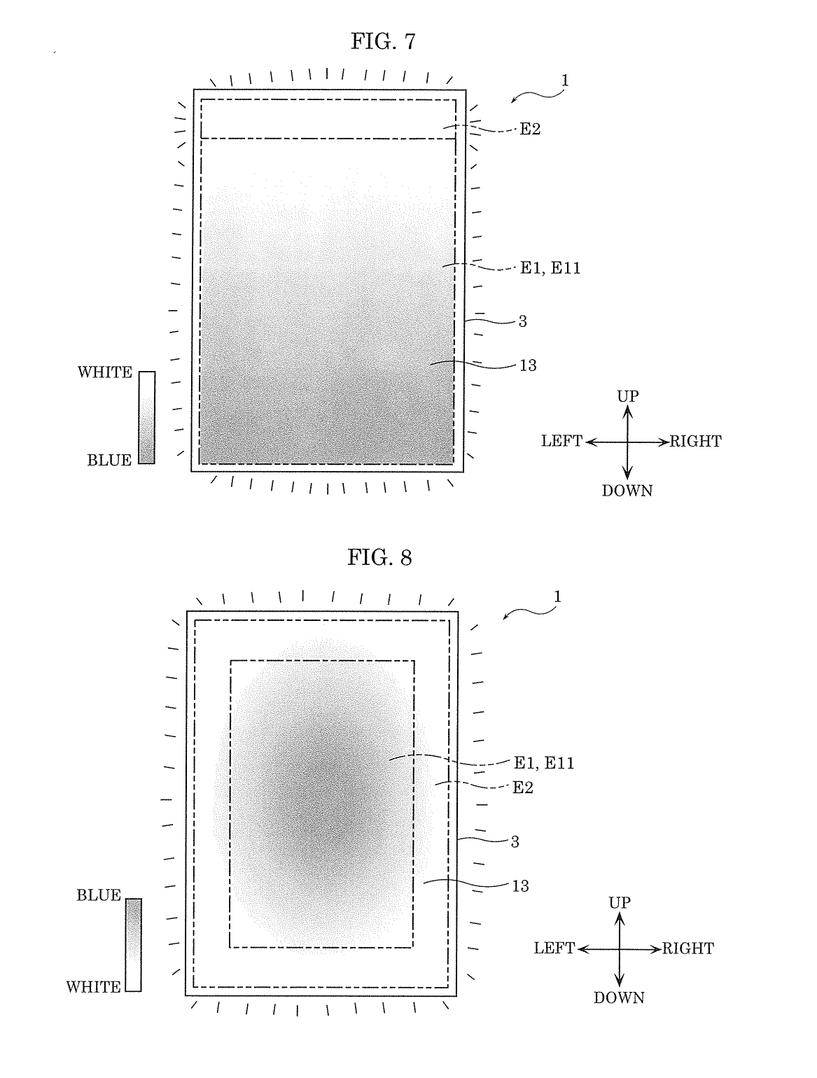

Light emission apparatus 1 according to Variation 1 of Embodiment 1 will be described with reference to FIG. 7.

FIG. 7 is a front view of light emission apparatus 1 according to Variation 1 of Embodiment 1.

Light emission apparatus 1 according to Embodiment 1 and light emission apparatus 1 according to Variation 1 of Embodiment 1 differ in that, as illustrated in FIG. 4, with light emission apparatus 1 according to Embodiment 1, light-emitting modules 9 are provided on the upper and lower sides of light emission apparatus 1, whereas with light emission apparatus 1 according to Variation 1 of Embodiment 1, light-emitting module 9 is only provided on the upper side, as illustrated in FIG. 7. In other words, as illustrated in FIG. 7, in Variation 1 of Embodiment 1, first light emission region E1 is the region excluding second light emission region E2 (the central and lower regions of light-transmissive plate 13). Moreover, light emission apparatus 1 according to Variation 1 of Embodiment 1 is similar to light emission apparatus 1 according to Embodiment 1, and as such, elements with like configurations share like reference signs and detailed description thereof will be omitted.

Second light emission region E2 is contiguous with a portion of an outer boundary of the light emission region. More specifically, second light emission region E2 is located in the upper portion of the light emission region, and first light emission region E1 is located below second light emission region E2.

Note that in Variation 1 of Embodiment 1, since light-emitting module 9 is provided on the upper side of light emission apparatus 1, second light emission region E2 is also located in the upper portion of light-transmissive plate 13, but the configuration is not limited to this example. For example, light-emitting module 9 may be provided along any given side, such as the bottom, right, or left side, and, accordingly, second light emission region E2 may be located along any given side, such as the bottom, right, or left side of light transmissive plate 13.

### (Advantageous Effects)

Next, the advantageous effects of light emission apparatus 1 according to Variation 1 of Embodiment 1 will be described.

As described above, in light emission apparatus 1 according to Variation 1 of Embodiment 1, second light emission region, E2 is contiguous with a portion of an outer boundary of the light emission region.

With this configuration, compared to light emission apparatus 1 in which two light-emitting modules 9 are disposed at two ends so as to oppose one another (light emission apparatus 1 according to Embodiment 1), costs can be cut. Moreover, if the user uses light emission apparatus 1, the user's eyes can be irradiated with blue and white light whereby the user's body can be activated.

Moreover, light emission apparatus 1 according to Variation 1 of Embodiment 1 achieves the same advantageous effects as light emission apparatus 1 according to Embodiment 1.

### Variation 2 of Embodiment 1

### (Configuration)

Light emission apparatus 1 according to Variation 2 of Embodiment 1 will be described with reference to FIG. 8.

FIG. 8 is a front view of light emission apparatus 1 according to Variation 2 of Embodiment 1.

Light emission apparatus 1 according to Embodiment 1 and light emission apparatus 1 according to Variation 2 of Embodiment 1 differ in that, as illustrated in FIG. 4, with light emission apparatus 1 according to Embodiment 1, light-emitting modules 9 are provided on the upper and lower sides of light emission apparatus 1, whereas with light emission apparatus 1 according to Variation 2 of Embodiment 1, light-emitting modules 9 are provided on inner surfaces of enclosure 3, as illustrated in FIG. 8. Moreover, light emission apparatus 1 according to Variation 2 of Embodiment 1 is similar to light emission apparatus 1 according to Embodiment 1, and as such, elements with like configurations share like reference signs and detailed description thereof will be omitted.

In light emission apparatus 1 according to Variation 2 of Embodiment 1, light-emitting modules 9 are disposed on the upper, lower, left, and right inner surfaces of enclosure 3.

Second light emission region E2 surrounds first light emission region E1. In other words, second light emission region E2 is contiguous with all outer boundaries of the light emission region.

### (Advantageous Effects)

Next, the advantageous effects of light emission apparatus 1 according to Variation 2 of Embodiment 1 will be described.

As described above, in light emission apparatus 1 according to Variation 2 of Embodiment 1, second light emission region E2 surrounds first light emission region E1.

With this configuration, since first light emission region E1 is surrounded by second light emission region E2, even if the user's gaze moves away from first light emission region E1, the user's gaze can easily return to first light emission region E1, which is lower in luminance than second light emission region E2.

Moreover, light emission apparatus 1 according to Variation 2 of Embodiment 1 achieves the same advantageous effects as light emission apparatus 1 according to Embodiment 1.

### Embodiment 2

### (Configuration)

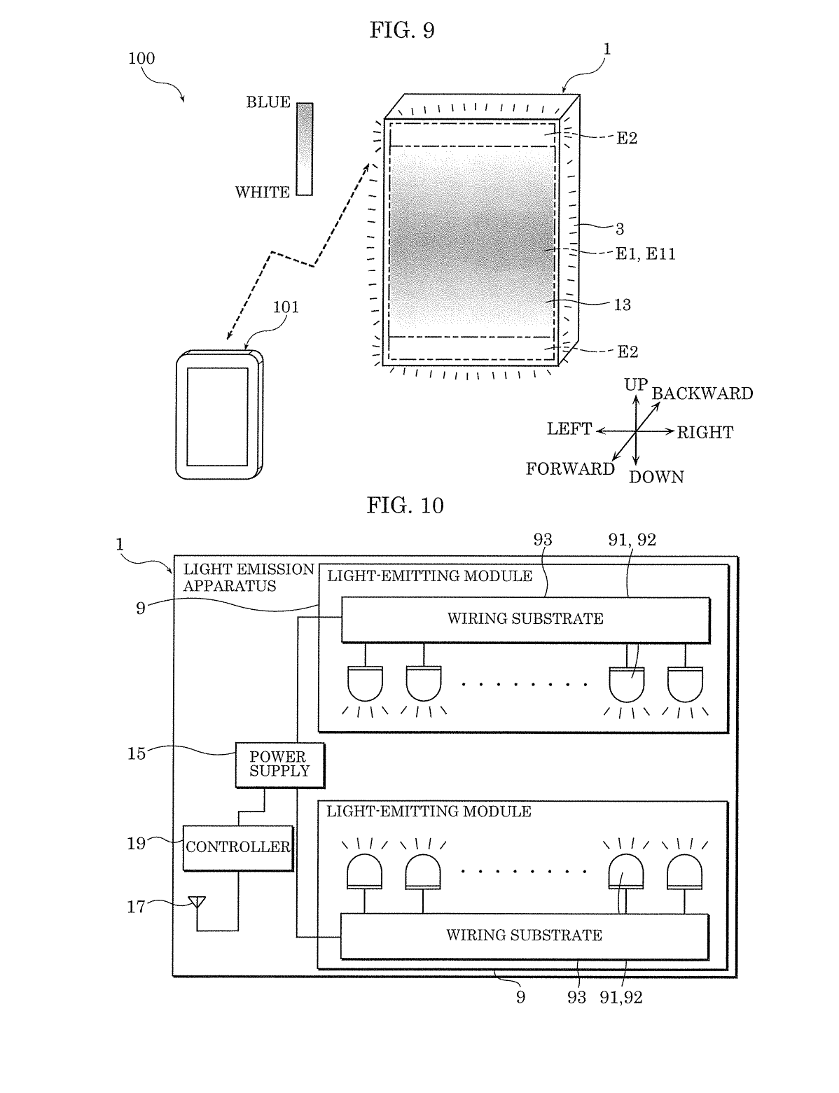

Light-emitting system 100 according to Embodiment 2 will with reference to FIG. 9 and FIG. 10.

FIG. 9 is a perspective view of light-emitting system 100 according to Embodiment 2. FIG. 10 is a block diagram of light emission apparatus 1 in the light-emitting system according to Embodiment 2.

Light emission apparatus 1 according to Embodiment 2 is similar to light emission apparatus 1 according to Embodiment 1, and as such, elements with like configurations share like reference signs and detailed description thereof will be omitted.

As illustrated in FIG. 9, light-emitting system 100 includes terminal apparatus 101 and light emission apparatus 1.

Terminal apparatus 101 is, for example, a remote control, a smartphone, or a tablet terminal apparatus, and is a separate apparatus from light emission apparatus 1. Terminal apparatus 101 includes a transmitter that transmits a signal that controls the illumination state of light-emitting modules 9. In other words, when the user wants to turn on or off light emission apparatus 1, the user operates terminal apparatus 101 to transmit, to light emission apparatus 1 via the transmitter of terminal apparatus 101, a signal that controls the illumination state of light-emitting modules 9.

As illustrated in FIG. 10, light emission apparatus 1 includes, in addition to light-emitting modules 9 and power supply 15 according to Embodiment 1, receiver 17 and controller 19.

Receiver 17 is realized as a communications circuit, and includes a function of receiving, via an antenna, a signal wirelessly transmitted from terminal apparatus 101, and transmitting the signal to controller 19.

Controller 19 controls the illumination state of light-emitting modules 9 in accordance with the signal received by receiver 17. More specifically, controller 19 controls, for example, operations such as the turning on or off, or dimming (brightness adjustment) of light-emitting modules 9, as well as color adjustment (adjustment of light emission color (color temperature)), in accordance with an instruction (a control signal from, for example, a remote control) from the user.

Controller 19 is configured of, for example, a circuit for controlling, for example, light-emitting modules 9. Controller 19 performs the above operations via, for example, a processor or microcomputer, or a dedicated. circuit.

Power supply 15 is electrically connected to, for example, light-emitting modules 9 and controller 19.

In light-emitting system 100, the user places light emission apparatus 1 on a table, and operates terminal apparatus 101 in order to perform operations such as the turning on or off of light emission apparatus 1. The user bathes in the blue and white light emitted from the light emission region of light emission apparatus 1.

### (Advantageous Effects)

Next, the advantageous effects of light-emitting system 100 according to Embodiment 2 will be described.

As described above, light-emitting system 100 according to Embodiment 2 includes: terminal apparatus 101 that transmits a signal; and light emission apparatus 1 that emits light that activates a human body. Light emission apparatus 1 includes: light-emitting module 9 that emits light from a light emission region; receiver 17 that receives the signal from terminal apparatus 101; and controller 19 that controls an illumination state of light-emitting module 9 in accordance with the signal received by receiver 17. The light emission region includes: first light emission region E1 that emits light having a first luminance; and second light emission region D2 that emits light having a second luminance greater than the first luminance, second light emission region E2 being a different region than first light emission region E1. First light emission region E1 is located in a central region of the light emission region, second light emission region E2 is peripheral to first light emission region E1, and the light having the first luminance emitted from first light emission region E1 has a predetermined wavelength that activates the human body.

Moreover, light-emitting system 100 according to Embodiment 2 achieves the same advantageous effects as Embodiment 1.

### Other Variations, etc.

Hereinbefore, the light emission apparatus, light emission method, and light-emitting system according to the present disclosure have been described based on Embodiments 1 and 2 and Variations 1 and 2 of Embodiment 1, but the present disclosure is not limited to Embodiments 1 and 2 and Variations 1 and 2 of Embodiment 1.

For example, in the above embodiments, in a front view of the light emission apparatus (when the light emission apparatus is viewed from the front), the light emission apparatus has a rectangular shape, but the shape of the light emission apparatus in this view is not limited to a rectangular shape. For example, in this view, the light emission apparatus may have a circular, polygonal such as triangular, or crescent shape, and may have a combination of these shapes. Moreover, for example, when the light emission apparatus has a circular shape in this view, the light-emitting module or modules may be provided across the inner circumferential surface of the enclosure, and, alternatively, a pair of light-emitting modules may be provided in locations having point symmetry about the center of the circle. In other words, a pair of arc-shaped light-emitting modules may be provided in mutually symmetrical locations. Stated differently, the second light emission region may be contiguous with a portion of an outer boundary of the light emission region.

Moreover, in the above embodiments, all of the plurality of light sources may be disposed on the bottom of the enclosure such that their optical axes all extend forward. In this case, the output of the power supply may be controlled so as to reduce the luminance (first luminance) of the light sources corresponding to the first light emission region.

Moreover, in Embodiment 1, the second light emission regions are located in upper and lower sides, but this example is not limiting; the second light emission regions may be located in left and right sides. In this case, the light-emitting modules are also provided on the left and right sides in the enclosure.

Moreover, in the above embodiments, at any given time only the blue light sources may be turned on, and, alternatively, only the white light sources may be turned on. In other words, it is not necessary that both the blue and white light sources be on at the same time. In a state in which only the blue light sources are turned on, the white light sources may be capable of being turned on in accordance with an operation made by the user, and vice versa. Moreover, the turned on blue light sources may be turned off at the same time as the turned off white light sources are turned on and vice versa (the turning on and off of the blue and white light sources can be performed alternately).

Moreover, in the above embodiments, when only either the white or blue light sources are on, a gradation region may be provided in which luminance gradually changes. In other words, the gradation region may be realized as a region which the luminance of a single color changes.

Moreover, in the above embodiments, a control is electrically connected to the light emission apparatus, but the control may be a remote control capable of operating the light emission apparatus (turning on and off the power, for example) via wireless communication. The wireless communication is realized by including a communication unit in the light emission apparatus performs wireless communication with the remote control. The communication unit may be a device having a near-field communication function, such as ZigBee (registered trademark), Wi-Fi (registered trademark), or Bluetooth (registered trademark).

While the foregoing has described one or more embodiments and/or other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present teachings.

What is claimed is:

1. A light-emitting apparatus that emits light that activates a human body, the light-emitting apparatus comprising
a light-emitting module that emits light from a light emission region,
wherein the light emission region includes:
a first light emission region that emits light having a first luminance; and
a second light emission region that emits light having a second luminance greater than the first luminance, the second light emission region being a different region than the first light emission region,
the first light emission region is located in a central region of the light emission region,
the second emission region is peripheral to the first light emission region, and
the light having the first luminance emitted from the first light emission region has a predetermined wavelength that activates the human body.

2. The light-emitting apparatus according to claim 1, wherein color of the light from the first emission region is blue and color of the light from the second emission region is white.

3. The light-emitting apparatus according claim 1, wherein
the second light emission region comprises a plurality of regions that sandwich the first light emission region.

4. The light-emitting apparatus according to claim 1, wherein
the second light emission region surrounds the first light emission region.

5. The light-emitting apparatus according to claim 1, wherein
the second light emission region is contiguous with a portion of an outer boundary of the light emission region.

6. The light-emitting apparatus according to claim 1, wherein
the light-emitting module includes a pair of light-emitting nodules,
the light-emitting apparatus further comprises:
an enclosure that houses the pair of light-emitting modules; and
a light-transmissive plate that includes the light emission region and covers an opening in the enclosure,
wherein each light-emitting module in the pair of light-emitting modules includes a white light source and a blue light source,
in the enclosure, a first light-emitting module among the pair of light-emitting modules and a second light-emitting module among the pair of light-emitting modules face each other, and
the blue light sources are disposed closer to a bottom of the enclosure than the white light sources are.

7. The light-emitting apparatus according to claim 6, further comprising
a diffusion sheet that is disposed on an inner surface of the enclosure and diffuses light.

8. The light-emitting apparatus according to claim 1, wherein
the first luminance of the first light emission region is less than 23000 cd/m², and
the second luminance of the second light emission region is at least 23000 cd/m².

9. The light-emitting apparatus according to claim 1, wherein:
the predetermined wavelength includes a light emission peak of the light emitting module in a range from 430 nm to 495 nm,
the first luminance of the first light emission region is at least 1000 cd/m² and less than 23000 cd/m², and
the second luminance of the second light emission region is at least 23000 cd/m² and at most 74000 cd/m².

10. The light-emitting apparatus according to claim 1, wherein
the first light emission region includes a gradation region, and
in the gradation region, the first luminance of the first light emission region gradually decreases with increasing distance from the second light emission region.

11. A light emission method of emitting light for light bathing using the light-emitting apparatus according to claim 1, comprising
irradiating a human eye with light from the light emission region, the irradiating being performed by the light-emitting apparatus.

12. A light-emitting system, comprising:
a terminal apparatus that transmits a signal; and
a light-emitting apparatus that emits light that activates a human body,
wherein the light-emitting apparatus includes:
a light-emitting module that emits light from a light emission region;
a receiver that receives the signal from the terminal apparatus; and
a controller that controls an illumination state of the light-emitting module in accordance with the signal received by the receiver,
the light emission region includes:
a first light emission region that emits light having a first luminance; and
a second light emission region that emits light having a second luminance greater than the first luminance, the second light emission region being a different region than the first light emission region,
the first light emission region is located in a central region of the light emission region,
the second light emission region is peripheral to the first light emission region, and
the light having the first luminance emitted from the first light emission region has a predetermined wavelength that activates the human body.
